(19) United States
(12) Patent Application Publication
(10) Pub. No.: US 2014/0296528 A1
(43) Pub. Date: Oct. 2, 2014

(54) 4-ARYL-1-(BIARYLMETHYLENE) PIPERIDINE COMPOUNDS

(71) Applicants: KING ABDULAZIZ CITY FOR SCIENCE AND TECHNOLOGY, Riyadh (SA); KING FAHD UNIVERSITY OF PETROLEUM AND MINERALS, Dhahran (SA)

(72) Inventors: NISAR ULLAH, DHAHRAN (SA); ALI AHMED AL-SHAHERI, RADA'A (YE)

(73) Assignees: KING ABDULAZIZ CITY FOR SCIENCE AND TECHNOLOGY, Riyadh (SA); KING FAHD UNIVERSITY OF PETROLEUM AND MINERALS, Dhahran (SA)

(21) Appl. No.: 13/852,998

(22) Filed: Mar. 28, 2013

(51) Int. Cl. C07D 401/04; C07D 401/14

(52) U.S. Cl. CPC C07D 401/04; C07D 401/14
USPC 546/157

(57) ABSTRACT

The 4-aryl-1-(biarylmethylene)piperidine compounds are piperadine compounds having a biaryl substituent linked to the nitrogen atom of the piperidine ring by a methylene (—CH₂—) group, and an aryl moiety attached to the 4-position of the piperidine ring. The aryl moiety may be a methoxy quinoline group, a 2-oxo quinoline group, or a 2-oxo, 3,4-dihydroxy quinoline group. The biaryl substituent may be biphenyl, fluorophenyl benzene, 3-phenyl pyridine, 3-(4-fluorophenyl) pyridine, phenyl cyclopentene, or 3-(1-cyclopenten-1-yl) pyridine. The compounds are believed to be suitable for anti-psychotic medication, since they are structurally related to SLV-313, a potential atypical antipsychotic having potent D₂ receptor antagonist and 5-HT_{1A} receptor agonist properties.

### BACKGROUND OF THE INVENTION

1. Field of the Invention

The present invention relates to piperidine compounds, and particularly to 4-aryl-1-(biarylmethylene)piperidine compounds, which are used for treating schizophrenia and related disorders.

2. Description of the Related Art

Schizophrenia is a complex lifelong chronic neuropsychiatric illness, afflicting approximately 1% of the world population. The symptoms of the disease can be grouped as positive and negative. Positive symptoms include delusions, hallucinations, and conceptual disorganization. The most characteristic negative symptoms are affective flattening, social withdrawal, anhedonia, and poverty of thought and content of speech. The typical antipsychotic drugs, for example, haloperidol or chlorpromazine, were the most widely used drugs, for they block D₂ receptors. However, although the blockade of D₂ receptors improves the positive symptoms, the development of neurological side effects, such as dystonia, muscle rigidity, tremor and akathisia, and tardive dyskinesia undermine compliance, in particular, extrapyramidal side effects (EPS). Various atypical or second-generation antipsychotics, such as clozapine, and more recently, aripiprazole, have been developed to reduce EPS liability and to treat negative symptoms. The atypical antipsychotics combine D₂ receptor antagonism with activity at other receptors, such as serotonergic receptors, on the premise that a suitable balance of pharmacological activity should broaden the spectrum of therapeutic efficacy and reduce EPS. It has been demonstrated that the combination of a dopamine D₂ receptor antagonist with 5-HT_{1A} receptor agonist properties could improve the therapeutic window, side-effect profile, and therapeutic efficacy of antipsychotic agents. As a result adoprazine

(having the formula shown in FIG. 5) (SLV-313) and bifeprunox (having the formula shown in FIG. 6), possessing potent D₂ receptor antagonist and 5-HT_{1A} receptor agonist properties, were developed. However, the failure of adoprazine (FIG. 5) and bifeprunox (FIG. 6) to oppose phencyclidine-induced social interaction deficits suggested that an appropriate ‘balance’ of activity at these sites is necessary for activity in this model. Thus, the need to discover compounds having varying ratios of D₂ and 5-HT_{1A} activities continues.

Thus, 4-aryl-1-(biarylmethylene)piperidine compounds solving the aforementioned problems is desired.

### SUMMARY OF THE INVENTION

The 4-aryl-1-(biarylmethylene)piperidine compounds have the formula:

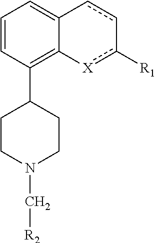

wherein R₁ is methoxy or oxo; X is N or NH; R₂ is a biaryl substituent selected from the group consisting of biphenyl, fluorophenyl benzene, 3-phenyl pyridine, 3-(4-fluorophenyl) pyridine, phenyl cyclopentene, and 3-(1-cyclopenten-1-yl) pyridine; and the dashed lines (------) represent either a single or a double bond; and pharmaceutically acceptable salts thereof. The compounds may be formulated as a tablet, capsule, or other pharmaceutical composition, together with appropriate excipients, such as binders, fillers, time release agents, etc.

It is believed that because of their structural similarity to adoprazine and bifeprunox, the compounds may be used as anti-psychotics for treating schizophrenia, schizoaffective disorder, and schizophreniform disorder, the method comprising administering to a patient in need thereof an effective amount of a 4-aryl-1-(biarylmethylene)piperidine compound or pharmaceutically acceptable salts thereof.

The 4-aryl-1-(biarylmethylene)piperidine compounds are structurally related to SLV-313 (adoprazine), an atypical antipsychotic having potent D₂ receptor antagonist and 5-HT_{1A} receptor agonist properties.

These and other features of the present invention will become readily apparent upon further review of the following specification and drawings.

### BRIEF DESCRIPTION OF THE DRAWINGS

FIG. 1 is a general structural formula for representative methoxy-substituted 4-aryl-1-(biarylmethylene)piperidine compounds according to the present invention, wherein R is either hydrogen or one of the six functional groups shown in FIG. 4.

FIG. 2 is a general structural formula for representative oxo-substituted (quinolin-2(1H)-one, having a double bond t to the oxo substitution) 4-aryl-1-(biarylmethylene)piperidine compounds according to the present invention, wherein R is either hydrogen or one of the six functional groups shown in FIG. 4.

FIG. 3 is a general structural formula for representative oxo-substituted (3,4-dihydroquinolin-2(1H)-one, having a single bond β to the oxo-substitution) 4-aryl-1-(biarylmethylene)piperidine compounds according to the present invention, wherein R is either hydrogen or one of the six functional groups shown in FIG. 4

FIG. 4 is the structural formulas for the R-group substituents for the compounds of FIGS. 1-3.

FIG. 5 is the structural formula of adoprazine, a compound of the prior art having potent D₂ receptor antagonist and 5-HT_{1A} receptor agonist properties.

FIG. 6 is the structural formula of bifeprunox, another compound of the prior art having potent D₂ receptor antagonist properties.

FIG. 7A is a reaction scheme for the synthesis of aldehydes 6c and 6d, which are intermediates in the synthesis of some of the 4-aryl-1-(biarylmethylene)piperidine compounds according to the present invention.

FIG. 7B is a reaction scheme for the synthesis of aldehydes 6e and 6f, which are intermediates in the synthesis of some of the 4-aryl-1-(biarylmethylene)piperidine compounds according to the present invention.

FIG. 8 is a reaction scheme for the synthesis of arylpiperidines 4 and 5 according to the present invention.

FIG. 9 is an alternative embodiment of a reaction scheme for the synthesis of arylpiperidines 4 and 5 according to the present invention.

FIG. 10 is a reaction scheme for the synthesis of arylpiperidine 3 according to the present invention.

FIG. 11 is a reaction scheme the synthesis of the 4-aryl-1-(biarylmethylene)piperidine compound 3a according to the present invention.

Similar reference characters denote corresponding features consistently throughout the attached drawings.

### DETAILED DESCRIPTION OF THE PREFERRED EMBODIMENTS

The 4-aryl-1-(biarylmethylene)piperidine compounds have the following general formula:

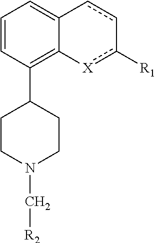

wherein R₁ is methoxy or oxo; X is N or NH; R₂ is a biaryl substituent selected from the group consisting of biphenyl (substituent (a) of FIG. 4), fluorophenyl benzene (substituent (b) of FIG. 4), 3-phenyl pyridine (substituent (c) of FIG. 4), 3-(4-fluorophenyl) pyridine (substituent (d) of FIG. 4), phenyl cyclopentene (substituent (e) of FIGS. 4), and 3-(1-cyclopenten-1-yl) pyridine (substituent (f) of FIG. 4); and the dashed lines (------) represent either a single or a double bond; and pharmaceutically acceptable salts thereof. The compounds may be formulated as a tablet, capsule, or other pharmaceutical composition, together with appropriate excipients, such as binders, fillers, time release agents, etc.

It is believed that because of their structural similarity to adoprazine and bifeprunox, the compounds may be used as anti-psychotics for treating schizophrenia, schizoaffective disorder, and schizophreniform disorder, the method comprising administering to a patient in need thereof an effective amount of the 4-aryl-1-(biarylmethylene)piperidine compounds or pharmaceutically acceptable salts thereof.

The 4-aryl-1-(biarylmethylene)piperidine compounds are structurally related to SLV-313 (adoprazine), an atypical antipsychotic having potent D₂ receptor antagonist and 5-HT_{1A} receptor agonist properties.

The 4-aryl-1-(biarylmethylene)piperidine compounds of the present invention were synthesized by the following reactions. Suzuki-Miyaura reaction of cyclic vinyl boronates, derived from the vinyl triflates of N-protected tetrahydropyridines, with appropriate aryl halides yielded 4-arylpiperidines. The reductive amination of the latter with suitable biarylaldehdyes accomplished the synthesis of the 4-aryl-1-(biarylmethylene)piperidine compounds according to the present invention.

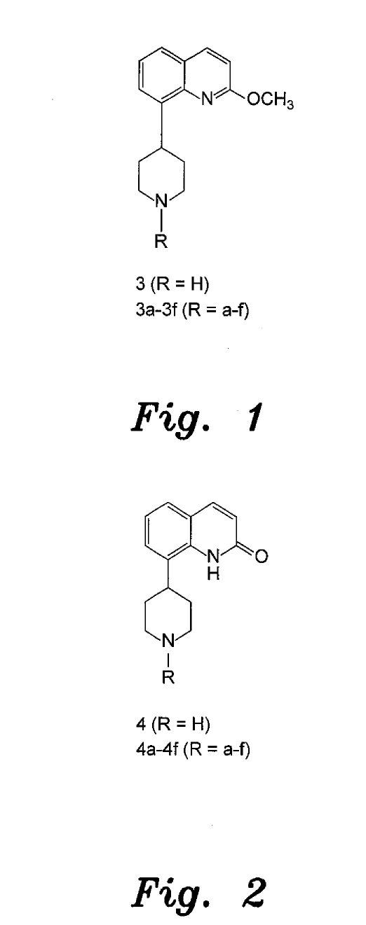

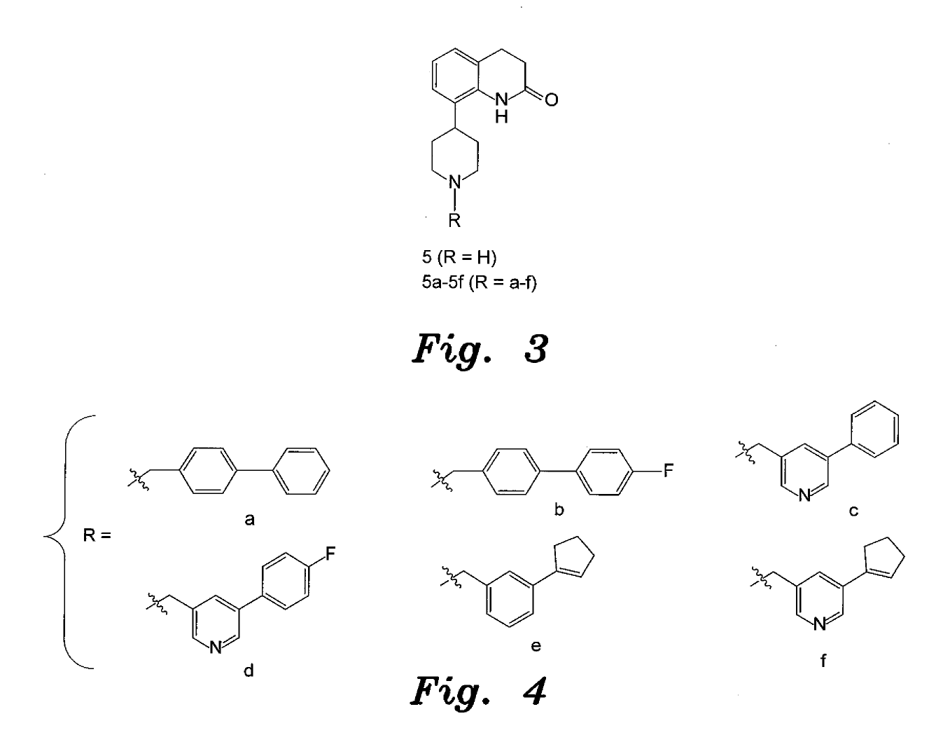

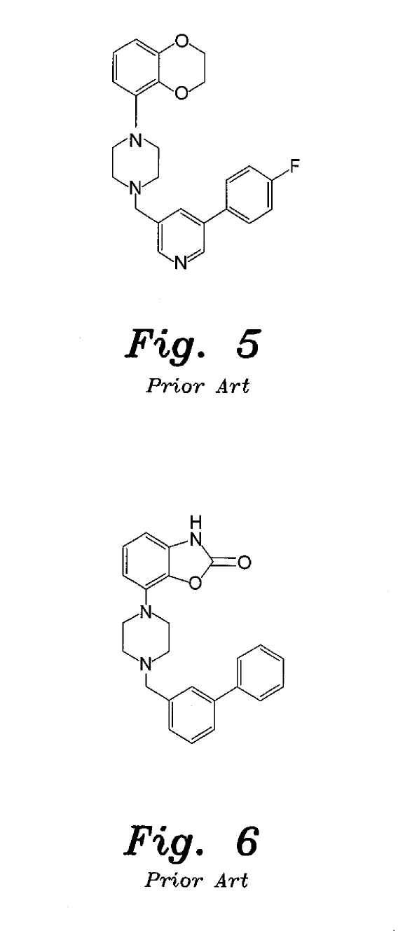

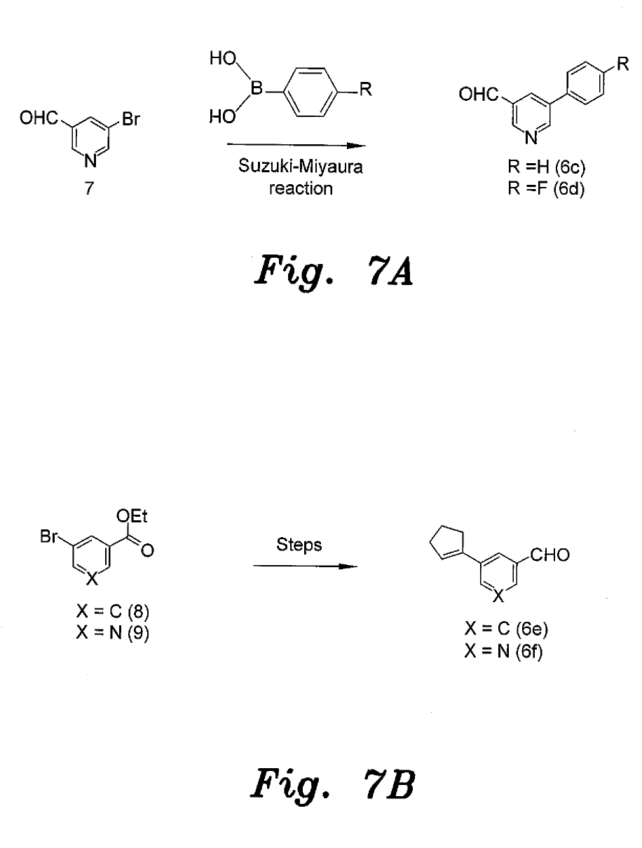

The synthesis of compounds 3a-3f (the base compound shown in FIG. 1 with the substituents (a) through (f) of FIG. 4), 4a-4f (the base compound shown in FIG. 2 with the substituents (a) through (f) of FIGS. 4) and 5a-5f (the base compound shown in FIG. 3 with the substituents (a) through (f) of FIG. 4) required the synthesis of aldehydes 6a-6f. Suzuki reaction of 4-bromobenzaldehyde with phenylboronic acid yielded 6a, while Suzuki reaction of 4-bromobenzaldehdye with 4-fluoroboronic acid yielded 6b. Reaction between 5-bromonicotinaldehyde 7 with the appropriate boronic acid gave the desired aldehydes 6c and 6d, respectively, as shown in FIG. 7A. The known aldehydes 6e and 6f were synthesized from their corresponding bromides 8 and 9 by employing literature known procedures described in S. Cuisiat et al., J. Med. Chem. (2007), Vol. 50, pp. 865-876, and as shown in Scheme 1 of FIG. 7B.

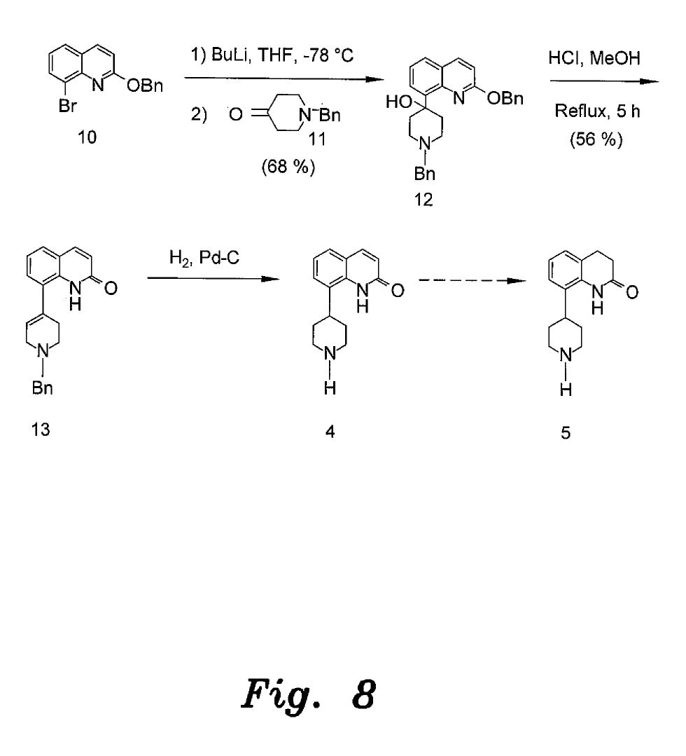

As shown in FIG. 8, the synthesis of the required arylpiperidines was commenced from lithiation of 10 in THF at −78° C., followed by quenching it with N-protected piperidinone 11 to obtain alcohol 12 in 68% yield. The dehydration of latter was ensued by refluxing it in concentrated HCl and MeOH to generate compound 13 in a moderate yield. To produce the desired intermediate 4 from compound 13, removal of N-protection and reduction of the double bond were required. Hence, compound 13 was subjected to hydrogenation in a Parr apparatus at 60 psi for 5 hours. The benzyl deprotection, however, proved to be stubborn, and the reaction yielded a very polar mixture of products, which were difficult to separate.

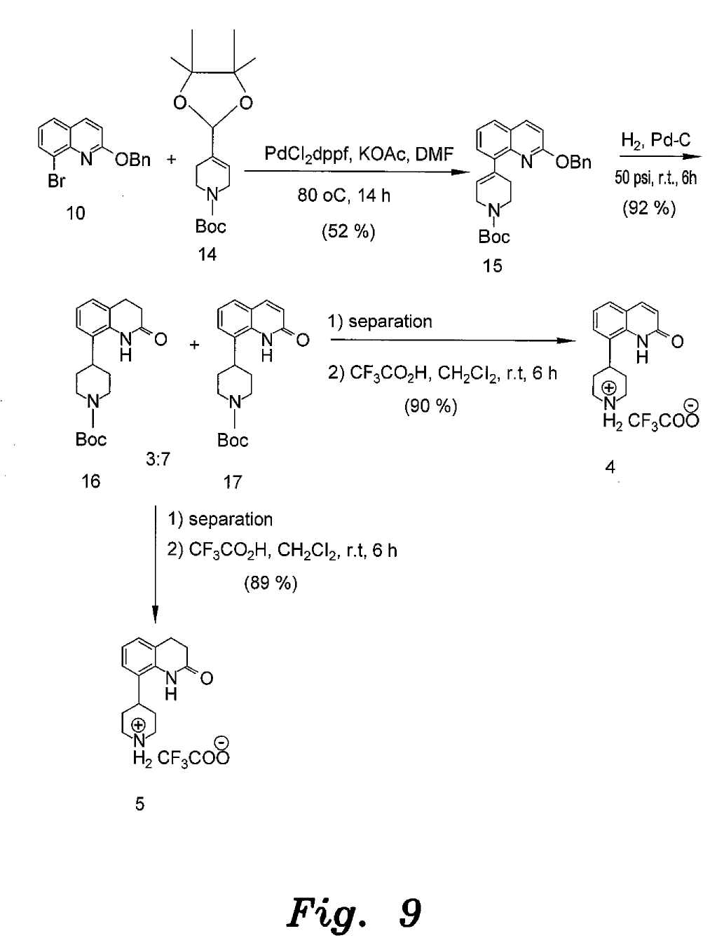

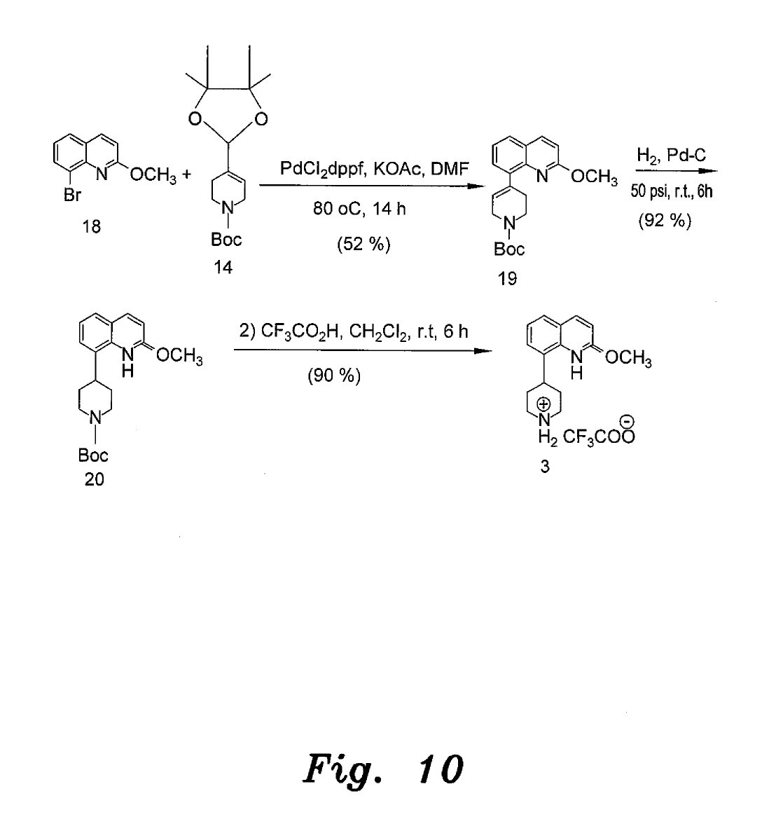

Thus, the desired intermediates 4 and 5 were synthesized from an alternative route, as outlined in Scheme 3 of FIG. 9. Suzuki-Miyaura reaction of cyclic vinyl boronates 14, derived from the vinyl triflates of N-protected tetrahydropyridines, with bromoquinoline 10 generated compound 15. Hydrogenation of the intermediate 15 in a Parr apparatus at 50 psi for 6 hours, followed by column chromatographic purifications on silica gel, produced intermediates 16 and 17 in 3:7 ratio. Exposure of compounds 16 and 17 to trifluoroacetic acid at room temperature smoothly furnished the desired intermediates 4 and 5 in high yields.

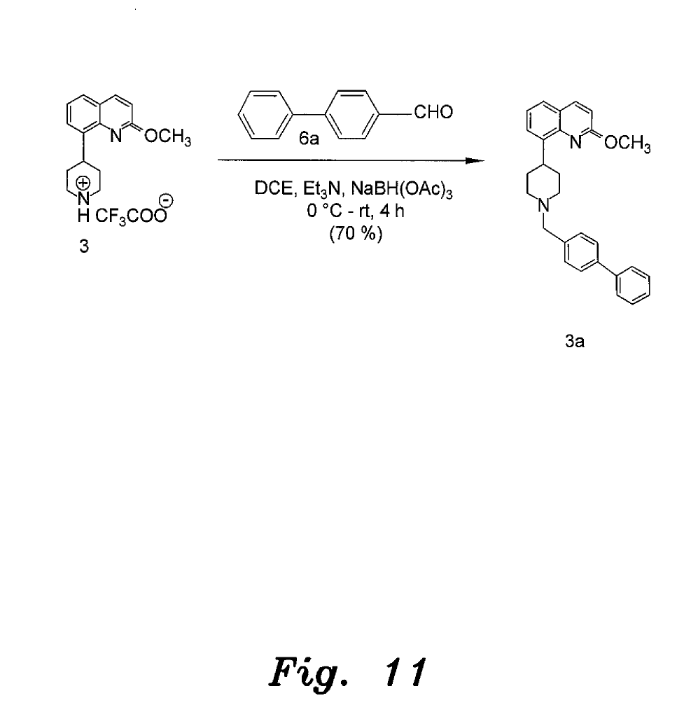

As shown in FIG. 10, to synthesize the required intermediate 3, Suzuki-Miyaura reaction of cyclic vinyl boronates 14 with bromoquinoline 18 generated compound 19, which, in turn, was hydrogenated at 50 psi for 7 h to furnish the intermediate 20. Exposure of the latter to trifluoroacetic acid at room temperature smoothly produced the desired intermediate 3 in an overall yield of 36% from 18 as shown in Scheme 4 of FIG. 10.

Having the desired arylpiperidines (4-5) and biarylaldehdyes (6a-6f) in hand, we next performed the reductive amination of arylpiperidines and aldehydes in 1,2-dichloroethane, using NaBH(OAc)₃ as reducing agent to accomplish the final compounds (3a-3f, 4a-4-f and 5a-5f) as shown in a representative Example in Scheme 5 of FIG. 11.

The following Examples illustrate the preparation of compounds of the invention.

### Example 1

### 1-Benzyl-4-(2-(benzyloxy)quinolin-8-yl)piperidin-4-ol (Compound 12)

A solution of 2-(benzyloxy)-8-bromoquinoline 10 (2.0 g, 6.4 mmol) in THF (20 mL) was added dropwise over 10 min to a solution of n-BuLi (2.5 M, 2.8 mL, 7 mmol) in hexane cooled to −78° C. The mixture was stirred for 1 h at −78° C., and a solution of 1-benzylpiperidone 11 (1.21 g, 6.4 mmol) in THF (10 mL) was added dropwise over a period of 10 min, maintaining the reaction temperature at −78° C. The resulting mixture was stirred at −78° C. for 0.5 h, and at −10° C. for 1.5 h, whereupon a saturated solution of ammonium chloride (4 mL) was added. The reaction mixture was stirred and warmed to room temperature. Water (50 mL) was added to the reaction mixture and extracted with dichloromethane (3×30 mL). The combined organic extracts were washed with water, dried with Na₂SO₄, and filtered. The solvent was removed under reduced pressure, and the crude product was purified by flash chromatography (1M NH₃ in MeOH/dichloromethane, 2:98 to 7:93) to afford the title compound as dark brown thick oil (1.84 g 68%). IR (neat): ν=3365, 3042, 3032, 2971, 1607, 1485, 1260, 1192 cm⁻¹.—¹H NMR (500 MHz, CDCl₃): δ=2.15 (br. s, 4H, piperidine H), 2.73-2.78 (m, 5H, piperidine H, OH), 3.62 (s, 2H, NCH₂), 5.43 (s, 2H, OCH₂), 6.99 (d, J=9.0 Hz, 1H, 3-H), 7.23-7.47 (m, 11H, aromatic H), 7.60 (m, 2H, aromatic H), 8.04 (d, J=8.5 Hz, 1H, 4-H).—¹³C NMR (125.7 MHz, CDCl₃): δ=37.26, 49.46 (all Cₚᵢₚₑᵣ), 53.02 (OCH₂), 63.52 (NCH₂), 68.48 (Cₚᵢₚₑᵣ), 113.21 (C-3), 124.75, 126.30, 127.48, 127.80, 128.05, 128.54, 128.70, 128.87, 129.08, 129.39, 129.93, 136.84, 141.18, 142.25, 144.64 (all Cₐᵣₒₘ), 160.31 (C-2). C₄₂H₄₄N₄O₃ (652.82):calcd. C, 77.27; H, 6.79; N, 8.58. found C, 77.20; H, 6.84; N, 8.51.

### Example 2

### 8-(1-Benzyl-1,2, 3,6-tetrahydropyridin-4-yl)quinolin-2(1H)-one (Compound 13)

A solution of compound 12 (1.5 g, 5.35 mmol) in a mixture of methanol (15 mL) and concentrated HCl (15 mL) was heated at reflux temperature for 5 h. The reaction mixture was cooled, and the solvent was removed under reduced pressure to give crude product as a hydrochloride salt, which was converted to the free base (aq NaOHlethyl acetate) and purified by column chromatography, eluting with ethyl acetatehexane (20:80 to 40:60) to afford the title compound as a light yellow gum (0.95 g, 56%). IR (neat): ν=3182, 3054, 3022, 2978, 1638, 1610, 1465 cm⁻¹.—¹H NMR (500 MHz, CDCl₃): δ=2.15 (br. s, 2H, piperidine H), 2.97 (t, J=5.5 Hz, 2H, piperidine H), 3.34 (br. s, 2H, piperidine H), 3.87 (s, 2H, NCH₂Ph), 5.79 (br. s, 1H, piperidine H), 6.62 (d, J=9.5 Hz, 1H, 3-H), 7.18 (t, J=7.5 Hz, 1H, aromatic H), 7.28-7.47 (m, 8H, aromatic H), 7.76 (d, J=9.5 Hz, 1H, 4-H), 10.17 (br. s, 1H, NHCO).—¹³C NMR (125.7 MHz, CDCl₃): δ=31.52, 48.1, 51.9, 115.82 (all Cₚᵢₚₑᵣ), 120.93, 123.73, 124.66, 127.58, 128.07, 128.66, 128.88, 129.49, 129.99, 134.85, 136.44, 141.10, 141.85 (all Cₐᵣₒₘ)_{,} 160.32 (C-2).—C₂₁H₂₀N₂O (316.40):calcd. C, 79.72; H, 6.37; N, 8.85. found C, 79.66; H, 6.41; N 8.80.

### Example 3

### tert-Butyl 4-(2-(benzyloxy)quinolin-8-yl)-5,6-dihydropyridine-1(2H)-carboxylate (Compound 15)

Nitrogen was flushed for 3 minutes in a flask containing a solution of the boronate 14 (1.39 g, 4.5 mmol), K₂CO₃ (1.86 g, 13.5 mmol) and bromide 10 (1.49 g, 4.74 mmol) in DMF (30 mL), followed by the addition of PdCl₂dppf (0.23 g, 0.28 mmol). The reaction mixture was heated to 80° C. and stirred under N₂ overnight, cooled to room temperature, and filtered through a pad of celite. The filtrate was added to ethyl acetate (50 mL) and washed successively with water (20 mL), brine (3×15 mL), dried over Na₂SO₄ and evaporated. Column chromatography of the brown oily material on silica gel, eluting with ethyl acetate:hexanes (10:90), and then changing to (25:75) gave the title compound as light yellow amorphous solid (0.97 g, 52%).—IR (neat): ν=3043, 3021, 2978, 1681, 1607, 1442, 1175 cm⁻¹.—¹H NMR (500 MHz, CDCl₃): δ=1.49 (s, 9H, OC(CH₃)₃), 2.76 (br. s, 2H, piperidine H), 3.68 (br. s, 2H, piperidine H), 4.13 (br. s, 2H, piperidine H), 5.49 (s, 2H, OCH₂Ph), 5.85 (br. s, 1H, piperidine H), 6.95 (d, J=8.8 Hz, 1H, aromatic H), 7.30-7.38 (m, 4H, aromatic H), 7.46-7.48 (m, 3H, aromatic H), 7.63 (dd, J=1.5, 7.9 Hz, 1H, aromatic H), 7.99 (d, J=8.8 Hz, 1H, aromatic H). —¹³C NMR (125.7 MHz, CDCl₃): δ=28.77 (OC(CH₃)₃), 30.18, 44.32, 67.73 (all Cₚᵢₚₑᵣ), 79.74 (OC(CH₃)₃), 113.18, 124.14, 125.55, 127.22, 128.04, 128.20, 128.66, 129.12, 137.51, 139.51, 140.12, 144.24 (all Cₐᵣₒₘ), 155.60 (C═O), 161.02 (Cₐᵣₒₘ).—C₂₆H₂₈N₂O₃ (416.51): calcd. C, 74.97; H, 6.78; N, 6.73. found C, 74.91; H, 6.83; N, 6.67.

### Example 4

### tert-Butyl 4-(2-oxo-1,2,3,4-tetrahydroquinolin-8-yl)piperidine-1-carboxylate (Compound 16)

To a solution of compound 15 (0.7 g, 1.68 mmol) in a mixture of THF (5 mL) and EtOH (10 mL) was added Pd—C (10% wet basis, 0.5 g), and the mixture was subjected to hydrogenation in a Parr apparatus at 50 psi for 6 hours. After being filtered through a pad of celite, the solution was concentrated to get brown oily material, which was resolved over silica column eluting with ethyl acetate:hexanes (30:70) and then changing to (60:40) to get compound 16 as an off-white solid (0.15 g, 27%). M.p. 132-134° C.—IR (neat): ν=3245, 3019, 2971, 1668, 1603, 1472 cm⁻¹.—¹H NMR (500 MHz, CDCl₃): δ=1.49 (s, 9H, OC(CH₃)₃), 1.61 (m, 2H, piperidine H), 1.69 (m, 2H, piperidine H), 2.61 (m, 2H, 4-H), 2.79 (m, 1H, piperidine H), 2.85 (br. s, 2H, piperidine H), 2.93 (m, 2H, 3-H), 4.28 (br. s, 2H, piperidine H), 6.97 (t, J=7.6 Hz, 1H, aromatic H), 7.04-7.09 (m, 2 El, aromatic H), 8.30 (br. s, 1H, NHCO).—¹³C NMR (125.7 MHz, CDCl₃): δ=26.00 (C-4), 28.45 (OC(CH₃)₃), 30.68 (C-3), 32.13, 35.62 (all Cₚᵢₚₑᵣ), 79.56 (OC(CH₃)₃), 123.18, 124.48, 124.75, 126.02, 130.65, 134.23 (all Cₐᵣₒₘ)_{,} 154.75 (C═O), 172.03 (C-2). C₁₉H₂₆N₂O₃ (330.42): calcd. C, 69.06, H, 7.93, N, 8.48. found C, 69.00; H, 7.98; N, 8.41.

### Example 5

### tert-Butyl 4-(2-oxo-1,2-dihydroquinolin-8-yl)piperidine-1-carboxylate (Compound 17)

The title compound 17 was obtained from the reaction described for compound 16 as a light yellow solid (0.36 g, yield 65%). M.p. 101-103° C.—IR (neat): ν=3172, 3031, 2965, 1645, 1603, 1461, 1112 cm⁻¹.—¹H NMR (500 MHz, CDCl₃): δ=1.50 (s, 9H, OC(CH₃)₃), 1.69 (m, 2H, piperidine H), 1.91 (m, 2H, piperidine H), 3.10 (br, s, 2H, piperidine H), 3.42 (m, 1H, piperidine H), 4.30 (br. s, 2H, piperidine H), 6.67 (d, J=9.5 Hz, 1H, 341), 7.20 (t, J=7.6 Hz, 1H, aromatic 1-1), 7.40-7.45 (m, 2H, aromatic H), 7.78 (t, J=7.6 Hz, 1H, 4-H).—¹³C NMR (125.7 MHz, CDCl₃): δ=28.48 (OC(CH₃)₃), 30.29, 32.33, 34.70 (all Cₚᵢₚₑᵣ)_{,} 79.49 (OC(CH₃)₃), 120.20, 121.24, 122.54, 123.84, 126.41, 127.76, 131.38, 135.78, 141.74 (all Cₐᵣₒₘ), 154.83 (C═O), 163.99 (Cₐᵣₒₘ.), 172.58 (C═O). —C₁₉H₂₄N₂O₃ (328.41): calcd. C, 69.49; H, 7.37; N, 8.53. found C, 69.45; H, 7.43; N, 8.46.

### Example 6

### 8-(Piperidin-4-yl)quinolin-2(1H)-one (Compound 4)

To a solution of 17 (0.5 g, 1.52 mmol) in CH₂Cl₂ (15 mL) was added trifluoroacetic acid (3 mL) at 0° C., and the reaction mixture was stirred for 6 h at room temperature. Solvents were evaporated under reduced pressure, and triturating with diethyl ether gave the title compound 4 as trifluoroacetic acid salt as an off-white solid (0.45 g, 90%). M.p. 256-258° C.—IR (neat): ν=3266, 3031, 3011, 2990, 1672, 1618, 1445 cm⁻¹.—¹H NMR (500 MHz, [D₆]DMSO): δ=1.86 (br. s, 4H, piperidine H), 3.08 (m, 2H, piperidine H), 3.42 (m, 2H, piperidine H), 3.48 (m, 1H, piperidine H), 6.51 (d, J=9.4 Hz, 1H, 3-H), 7.19 (t, J=8.1 Hz, 1H, aromatic H), 7.37 (d, J=8.0 Hz, 1H, aromatic H), 7.54 (d, J=7.9 Hz, 1 H, aromatic H H), 7.91 (d, J=9.5 Hz, 1H, 441), 8.49 (br. s, 1H, NHCO).—¹³C NMR (125.7 MHz, [D₆]DMSO): δ=28.91, 31.68, 44.05 (all Cₚᵢₚₑᵣ)_{,} 119.91, 121.60, 122.31, 127.09, 127.56, 129.40, 136.09, 141.55 (all Cₐᵣₒₘ), 162.92 (C═O).—C₁₆H₁₇F₃N₂O₃ (342.31): calcd. C, 56.14, H, 5.01, N, 8.18. found C, 56.08, H, 5.06, N, 8.11.

### Example 7

### 8-(Piperidin-4-yl)-3,4-dihydroquinolin-2(1H)-one (Compound 5)

Following the same procedure adopted for the synthesis of 4, the title compound was obtained from compound 16 as off-white solid (0.70 g, yield 89%). M.p. 247-248° C.—IR (neat): ν=3221, 3021, 2988, 1660, 1603, 1445, 1186 cm⁻¹.—¹H NMR (500 MHz, [D₆]DMSO): δ 1.79 (m, 4H, piperidine H), 2.51 (m, 2H, 2-H), 2.85 (m, 2H, 3-H), 2.99-3.06 (m, 3H, piperidine H), 3.34 (m, 2H, piperidine H), 6.96 (m, 1H, aromatic H), 7.07 (m, 1H, aromatic H), 9.63 (s, 1H, NHCO).—¹³C NMR (125.7 MHz, [D₆]DMSO): b=25.88 (C-4), 29.22 (Cₚᵢₚₑᵣ), 30.88 (C-3), 32.29, 44.82 (all Cₚᵢₚₑᵣ), 123.05, 124.68, 125.63, 126.62, 130.45, 135.37 (all Cₐᵣₒₘ), 170.02 (C═O). C₁₆H₁₉F₃N₂O₃ (344.33): calcd. C, 55.81, H, 5.56, N, 8.14. found C, 55.74; H, 5.62; N, 8.08.

### Example 8

### tert-Butyl 4-(2-methoxyquinolin-8-yl)-5,6-dihydropyridine-1(2H)-carboxylate (Compound 19)

Following the same procedure adopted for the synthesis of 15, the title compound was obtained from Suzuki reaction of boronate 14 and bromoquinoline 18 as dark brown gum, (0.64 g, yield 42%). M.p. 132-133° C.—IR (neat): ν=3037, 2978, 1677, 1604, 1486, 1176 cm⁻¹.—¹H NMR (500 MHz, CDCl₃): δ=1.49 (s, 9H, OC(CH₃)₃), 2.85 (br. s, 2H, piperidine H), 3.70 (m, 2H, piperidine H), 4.02 (s, 3H, OCH₃), 4.13 (br. s, 2H, piperidine H), 5.86 (br. s, 1H, piperidine H), 6.90 (d, J=8.5 Hz, 1H, 3-H), 7.32 (t, J=7.6 Hz, 1H, aromatic H), 7.48 (dd, J=1.5, 7.3 Hz, 1H, aromatic H), 7.63 (dd, J=1.2, 7.8 Hz, 1H, aromatic H), 7.97 (d, J=8.8 Hz, 1H, aromatic H).—¹³C NMR (125.7 MHz, CDCl₃): δ=24.54 (Cₚᵢₚₑᵣ), 28.51 (OC(CH₃)₃), 29.84, 42.32 (all Cₚᵢₚₑᵣ), 53.37 (OCH₃), 79.52 (OC(CH₃)₃), 112.71, 123.80, 125.13, 126.99, 128.81, 139.06, 139.74, 144.03, 161.37 (all Cₐᵣₒₘ).—C₂₀H₂₄N₂O₃ (340.42): calcd. C, 70.56; H, 7.11; N, 8.23. found C, 70.50; H, 7.16; N, 8.17.

### Example 9

### tert-Butyl 4-(2-methoxyquinolin-8-yl)piperidine-1-carboxylate (Compound 20)

To a solution of compound 19 (0.6 g, 1.76 mmol) in a mixture of THF (5 mL) and EtOH (10 mL) was added Pd—C (10% wet basis, 0.4 g), and the mixture was subjected to hydrogenation in a Parr apparatus at 60 psi for 7 hours. After filtering over the pad of celite, the solution was concentrated and chromatographed on silica column, eluting with ethyl acetate:hexanes (20:80) to get the title compound as off-white solid (0.57 g, yield 94%). M.p. 72-73° C.—IR (neat): ν=3031, 2935, 1675, 1608, 1484 cm⁻¹.—¹H NMR (500 MHz, CDCl₃): δ=1.42 (s, 9H, OC(CH₃)₃), 1.71 (m, 2H, piperidine H), 1.95 (m, 2H, piperidine H), 2.88 (m, 2H, piperidine H), 3.82 (m, 1H, piperidine H), 3.99 (s, 3H, OCH₃), 4.22 (br. s, 2H, piperidine H), 6.84 (d, J=8.5 Hz, 1H, 3-H), 7.28 (m, 1H, aromatic H), 7.38 (m, 1H, aromatic H), 7.51 (m, 1H, aromatic H), 7.97 (d, J=8.8 Hz, 1H, aromatic II).—¹³C NMR (125.7 MHz, CDCl₃): δ=28.48 (OC(CH₃)₃), 32.15, 36.84, 42.84 (all Cₚᵢₚₑᵣ), 53.23 (OCH₃), 79.30 (OC(CH₃)₃), 112.38, 123.89, 124.96, 125.64, 126.04, 139.40, 143.93 (all Cₐᵣₒₘ), 155.21 (C═O), 161.67 (C.). C₂₀H₂₆N₂O₃ (342.43): calcd. C, 70.15; H, 7.65; N, 8.18. found C, 70.10, H, 7.70, N, 8.11.

### Example 10

### 2-Methoxy-8-(piperidin-4-yl)quinoline (Compound 3)

Following the same procedure adopted for the synthesis of 4, the title compound was obtained from compound 20 as an off-white solid (0.46 g, yield 92%). M.p. 142-144° C.

IR (neat): ν=3031, 2982, 1612, 1441, 1213 cm⁻¹.—¹H NMR (500 MHz, [D₆]DMSO): δ=1.99 (m, 2H, piperidine H), 2.14 (m, 2H, piperidine H), 3.16 (m, 2H, piperidine H), 3.45 (m, 2H, piperidine H), 4.01 (s, 3H, OCH₃), 7.01 (d, J=8.2 Hz, 1H, 3-H), 7.41 (m, 1H, aromatic H), 7.51 (m, 1H, aromatic H), 7.92 (d, J=8.3 Hz, 1H, aromatic H).—¹³C NMR (125.7 MHz, [D₆]DMSO): S=28.72, 31.00, 34.40, 36.02, 44.33 (all Cₚᵢₚₑᵣ)_{,} 53.28 (OCH₃), 112.76, 114.52, 116.83, 124.23, 126.19, 126.48, 140.10, 158.76, 161.23 (all Cₐᵣₒₘ).—C₁₇H₁₉F₃N₂O₃ (356.34): calcd. C, 57.30; H, 5.37; N, 7.86. found C, 57.24; H, 5.42; N, 7.80.

### Example 11

### 8-(1-(Biphenyl-4-ylmethyl)piperidin-4-yl)-2-methoxyquinoline (Compound 3a)

To a solution of compound 3 (0.15 g, 0.42 mmol) and biphenyl-4-carbaldehyde 6a (0.1 g, 0.55 mmol) in 1,2-dichloroethane (5 mL) at 0° C. was added Et₃N (0.13 mL, 0.97 mmol). After being stirred for 10 min at room temperature, NaBH(OAc)₃ (0.11 g, 0.53 mmol) was added, and the reaction mixture was stirred for 6 h. To the reaction mixture was added sat. NaHCO₃ solution (10 mL) and stirred for 15 min, followed by the addition of ethyl acetate (30 mL). The organic layer was separated and washed with sat. NaHCO₃, brine, and dried over Na₂SO₄. Purification of the brown oily material on silica column, eluting with ethyl acetate:hexanes (70:30), and then changing to ethyl acetate (100%) yielded the titled compound 3a as a light yellow solid 0.126 g, yield 45%). M.p. 84-85° C.—IR (neat): ν=3031, 3021, 2936, 1609, 1444, 1186, 1120 cm⁻¹.—¹H NMR (500 MHz, CDCl₃): δ=2.15 (m, 2H, piperidine H), 2.36 (m, 2 II, piperidine H), 2.85 (br. s, 2H, piperidine H), 3.64 (m, 2H, piperidine H), 4.03 (s, 3H, OCH₃), 4.20 (s, 2H, NCH₂), 6.88 (d, J=8.8 Hz, 1H, 3-H), 7.32-7.38 (m, 2H, aromatic H), 7.45 (t, J=7.3 Hz, 2H, aromatic II), 7.51 (d, J=7.3 Hz, 1 aromatic H), 7.56-7.59 (m, 4H, aromatic H), 7.63 (m, 2H, aromatic H), 7.95 (d, J=8.5 Hz, 1H, aromatic H).—¹³C NMR (125.7 MHz, CDCl₃): δ=29.47, 34.92 (all Cₚᵢₚₑᵣ), 53.14 (OCH₃), 61.71 (NCH₂), 112.62 (C-3), 123.98, 124.98, 126.21, 127.06, 127.72, 128.85, 131.30, 139.31, 139.55, 140.02, 142.37, 143.86 (all Cₐᵣₒₘ), 161.44 (C-2). C₂₈H₂₈N₂O (408.53): calcd. C, 82.32, H, 6.91, N, 6.86. found C, 82.25; H, 6.96; N, 6.79.

### Example 12

### 8-(1-((4′-Fluorobiphenyl-4-yl)methylpiperidin-4-yl)-2-methoxyquinoline (Compound 3b)

Following the same procedure adopted for the synthesis of 3a, the title compound was obtained by reductive amination of compound 3 and 6b as an off-white solid (yield 37%). M.p. 94-95° C.—IR (neat): ν=3042, 3011, 2926, 1603, 1440, 1183, 1132 cm⁻¹.—¹H NMR (500 MHz, CDCl₃): δ=1.92 (m, 2H, piperidine 1-1), 2.02 (m, 2H, piperidine H), 2.30 (m, 2H, piperidine H), 3.18 (m, 2H, piperidine e H), 3.70 (s, 2H, NCH₂), 3.83 (m, 1H, piperidine H), 4.05 (s, 3H, OCH₃), 6.88 (d, J=8.5 Hz, 1H, 3-H), 7.09-7.12 (m, 2 II, aromatic H), 7.32 (t, J=7.6 Hz, 1H, aromatic H), 7.45-7.56 (m, 8H, aromatic H), 7.94 (d, J=8.8 Hz, 1H, aromatic H).—¹³C NMR (125.7 MHz, CDCl₃): δ=32.03, 36.07 (all Cₚᵢₚₑᵣ), 53.07 (OCH₃), 54.66 (Cₚᵢₚₑᵣ), 62.95 (NCH₂), 112.36 (C-3), 115.49, 115.66, 123.85, 124.91, 125.41, 125.81, 126.84, 128.52, 128.57, 129.97, 136.71, 136.98, 139.17, 142.32, 144.13 (all Cₐᵣₒₘ), 161.20 (C-2).—C₂₈H₂₇FN₂O (426.53): calcd. C, 78.85; H, 6.38; N, 6.57. found C, 78.79, H, 6.44, N, 6.50.

### Example 13

### 2-Methoxy-8-(1-((5-phenylpyridin-3-yl)methyl)piperidin-4-yl)quinoline (Compound 3c)

Following the same procedure adopted for the synthesis of 3a, the title compound was obtained by reductive amination of compound 3 and 6c as an off white solid (yield 35%). M.p. 135-137° C.—IR (neat): ν=3021, 2945, 1601, 1433, 1263, 1228 cm⁻¹.—¹H NMR (500 MHz, CDCl₃): δ=1.90 (m, 2H, piperidine H), 2.02 (m, 2H, piperidine H), 2.30 (m, 2H, piperidine H), 3.09 (m, 2H, piperidine H), 3.67 (s, 2H, NCH₂), 3.82 (m, 1H, piperidine H), 4.07 (s, 3H, OCH₃), 6.88 (d, J=8.8 Hz, 1H, 3-H), 7.33 (t, J=7.6 Hz, 1H, aromatic H), 7.41 (d, J=7.3 Hz, 1H, aromatic H), 7.47-7.53 (m, 3H, aromatic H), 7.64 (m, 2H, aromatic 1-1), 7.94 (m, 2H, aromatic H), 8.59 (d, J=1.8 Hz, 1H, aromatic H), 8.77 (d, J=2.1 Hz, 1H, aromatic H).—¹³C NMR (125.7 MHz, CDCl₃): δ=32.33, 36.36 (all Cₚᵢₚₑᵣ), 53.05 (OCH₃), 54.79 (Cₚᵢₚₑᵣ), 60.65 (NCH₂), 112.32 (C-3), 124.88, 125.33, 125.77, 127.16, 127.98, 128.97, 134.08, 135.05, 136.23, 137.81, 139.11, 142.49, 144.13, 146.95, 149.17 (all Cₐᵣₒₘ), 161.13 (C-2).—C₂₇H₂₇N₃O (409.52): calcd. C, 79.19; H, 6.65; N, 10.26. found C, 79.12; H, 6.71; N, 10.19.

### Example 14

### 8-(1-((5-(4-Fluorophenyl)pyridin-3-yl)methyl)piperidin-4-yl)-2-methoxyquinoline (Compound 3d)

Following the same procedure adopted for the synthesis of 3a, the title compound was obtained by reductive amination of compound 3 and 6d as a light brown solid (yield 32%). M.p. 156-158° C.—IR (neat): ν=3025, 2931, 1607, 1431, 1266 cm⁻¹.—¹H NMR (500 MHz, CDCl₃): δ=1.89 (m, 2H, piperidine H), 2.01 (m, 2H, piperidine H), 2.28 (m, 2H, piperidine H), 3.05 (m, 2H, piperidine H), 3.65 (s, 2H, NCH₂), 3.81 (m, 1H, piperidine H), 4.06 (s, 3H, OCH₃), 6.88 (d, J=8.5 Hz, 1H, 3-H), 7.16 (m, 2H, aromatic H), 7.33 (t, J=7.6, 1H, aromatic H), 7.51 (dd, J=1.2, 7.3 Hz, 1H, aromatic H), 7.54-7.59 (m, 3H, aromatic H), 7.89 (m, 1H, aromatic H), 7.94 (d, J=8.8 Hz, 1H, aromatic H), 8.59 (d, J=1.5 Hz, 1H, aromatic H), 8.77 (d, J=2.1 Hz, 1H, aromatic H).—¹³C NMR (125.7 MHz, CDCl₃): δ=32.33, 36.35 (all Cₚᵢₚₑᵣ), 53.04 (OCH₃), 54.82 (Cₚᵢₚₑᵣ.), 60.62 (NCH₂), 112.34 (C-3), 115.86, 116.03, 123.78, 124.90, 125.36, 125.77, 128.80, 128.85, 133.92, 134.22, 134.89, 135.33, 139.12, 142.47, 144.13, 146.76, 149.17, 161.13, 163.82 (all Cₐᵣₒₘ.). C₂₇H₂₆FN₃O (427.51): calcd. C, 75.85; H, 6.13; N, 9.83. found C, 75.79; H, 6.19; N, 9.76.

### Example 15

### 8-(1-(3-Cyclopentenylbenzyl)piperidin-4-yl)-2-methoxyquinoline (Compound 3e)

Following the same procedure adopted for the synthesis of 3a, the title compound was obtained by reductive amination of compound 3 and 6e as a light yellow solid (yield 46%). M.p. 125-126° C.—IR (neat): ν=3028, 2982, 2898, 1605, 1472, 1445, 1263, 1258 cm⁻¹.—¹H NMR (500 MHz, CDCl₃): δ=1.85-192 (m, 2H, piperidine H), 1.98-2.04 (m, 4H, piperidine H, cyclopent H), 2.24 (m, 2H, cyclopent H), 2.72 (m, 2H, cyclopent H), 3.05 (m, 2H, piperidine H), 3.60 (s, 2H, NCH₂), 3.79 (m, 1H, piperidine H), 4.05 (s, 3H, OCH₃), 6.20 (s, 1H, cyclopent H), 6.85 (d, J=8.8 Hz, 1H, 3-H), 7.24-7.34 (m, 4H, aromatic H), 7.45-7.54 (m, 3H, aromatic H), 7.91 (d, J=8.8 Hz, 1H, aromatic H).—¹³C NMR (125.7 MHz, CDCl₃): δ=23.33 (C_{cyclopent}), 32.33 (Cₚᵢₚₑᵣ), 33.22, 33.30 (all C_{cyclopent}), 36.32 (Cₚᵢₚₑᵣ), 53.05 (OCH₃), 54.74 (Cₚᵢₚₑᵣ), 63.62 (NCH₂), 112.34 (C_{cyclopent})_{,} 123.80, 124.27, 124.87, 125.28, 125.80, 126.12, 126.46, 127.88, 128.10, 136.67, 138.29, 139.11, 142.41, 142.67, 144.13, 161.12 (all Cₐᵣₒₘ).—C₂₇H₃₀N₂O (398.54): calcd. C, 81.37; H, 7.59; N, 7.03. found C, 81.31, H, 7.64, N, 6.97.

### Example 16

### 8-(1-((5-Cyclopentenylpyridin-3-yl)methyl)piperidin-4-yl)-2-methoxyquinoline (Compound 30

Following the same procedure adopted for the synthesis of 3a, the title compound was obtained by reductive amination of compound 3 and 6f as a light yellow amorphous solid (yield 39%). M.p. 85-86° C.—IR (neat): ν=3021, 2992, 2828, 1601, 1472, 1445, 1255 cm⁻¹.—¹H NMR (500 MHz, CDCl₃): δ=1.89-194 (m, 2H, piperidine H), 2.02-2.09 (m, 4H, piperidine H, cyclopent 11), 2.26-2.31 (m, 2H, cyclopent H), 2.73 (m, 2H, cyclopent H), 3.09 (m, 2H, piperidine H), 3.61 (s, 2H, NCH₂), 3.80 (m, 1H, piperidine H), 4.05 (s, 3H, OCH₃), 6.30 (s, 1H, cyclopent H), 6.87 (d, J=8.5 Hz, 1H, 3-H), 7.32 (m, 1H, aromatic H), 7.49 (d, J=7.3 Hz, 1H, aromatic H), 7.55 (d, J=7.9 Hz, 1H, aromatic H), 7.76 (s, 1H, aromatic H), 7.95 (d, J=8.8 Hz, 1H, aromatic H), 8.42 (s, 1H, aromatic H), 8.59 (s, 1H, aromatic H).—¹³C NMR (125.7 MHz, CDCl₃): δ23.16 (C_{cyclopent}), 32.09 (Cₚᵢₚₑᵣ) 32.88, 33.36 (all C_{cyclopent}), 36.24 (Cₚᵢₚₑᵣ), 53.03 (OCH₃) 54.57 (Cₚᵢₚₑᵣ), 60.46 (NCH₂), 112.32 (C_{cyclopent}), 123.77, 124.87, 125.36, 125.78, 128.35, 132.04, 133.14, 133.59, 139.10, 139.38, 142.34, 144.10, 145.74, 148.46 (all Cₐᵣₒₘ), 161.14 (C-2). C₂₆H₂₉N₃O (399.53): calcd. C, 78.16, H, 7.32, N, 10.52. found C, 78.10; H, 7.38; N, 10.45.

### Example 17

### 8-(1-(Biphenyl-4-ylmethyl)piperidin-4-yl)quinolin-2(1H)-one (Compound 4a)

Following the same procedure adopted for the synthesis of 3a, the title compound was obtained by reductive amination of compound 4 and 6a as a light yellow solid (yield 45%). M.p. 146-148° C.—IR (neat): ν=3193, 3038, 3021, 2938, 1641, 1601, 1437, 1218 cm⁻¹.—¹H NMR (500 MHz, CDCl₃): δ=1.81 (m, 4H, piperidine H), 2.26 (m, 2H, piperidine H), 3.00 (br. s, 3H, piperidine H), 3.60 (s, 2H, NCH₂), 6.65 (d, J=9.4 Hz, 1H, 3-H), 7.10 (t, J=8.7 Hz, 1H, aromatic H), 7.34-7.55 (m, 5H, aromatic H), 7.61 (d, J=9.4 Hz, 1H, 4-H), 10.12 (br. s, 1H, NHCO).—¹³C NMR (125.7 MHz, CDCl₃): δ=32.41, 34.93, 53.83 (all Cₚᵢₚₑᵣ), 63.03 (NCH₂), 119.99, 121.17, 122.47, 126.18, 126.92, 127.03, 127.16, 127.78, 128.73, 129.75, 131.41, 135.71, 137.02, 139.95, 140.92, 141.61 (all Cₐᵣₒₘ), 163.46 (C═O). C₂₇H₂₆N₂O (394.51): calcd. C, 82.20; H, 6.64; N, 7.10. found C, 82.14; H, 6.70; N, 7.03.

### Example 18

### 8-(1-((4′-Fluorobiphenyl-4-yl)methyl)piperidin-4-yl)quinolin-2(1H)-one (Compound 4b)

Following the same procedure adopted for the synthesis of 3a, the title compound was obtained by reductive amination of compound 4 and 6b as an off white solid (yield 41%). M.p. 159-161° C.—IR (neat): ν=3213, 3028, 2928, 1637, 1609, 1447, 1171 cm⁻¹.—¹H NMR (500 MHz, CDCl₃): δ=1.88 (m, 4H, piperidine H), 2.24 (m, 2H, piperidine H), 2.90 (m, 1H, piperidine H), 3.08 (m, 2H, piperidine H), 3.65 (s, 2H, NCH₂), 6.62 (d, J=9.4 Hz, 1H, 3-H), 7.12 (t, J=8.7 Hz, 1H, aromatic II), 7.20 (t, J=8.5 Hz, 1H, aromatic H), 7.41-7.44 (m, 4H, aromatic H), 7.52-7.58 (m, 5H, aromatic H), 7.73 (d, J=9.4 Hz, 1H, 4-H), 9.55 (br. s, 1H, NHCO).—¹³C NMR (125.7 MHz, CDCl₃): δ=32.32, 35.36, 54.03 (all Cₚᵢₚₑᵣ), 63.02 (NCH₂), 115.52, 115.73, 120.06, 121.21, 122.62, 126.32, 126.86, 127.82, 128.56, 128.64, 129.77, 130.08, 135.66, 137.05, 137.07, 139.03, 141.70 (all Cₐᵣₒₘ), 161.32 (C═O), 163.11 (Cₐᵣₒₘ).—C₂₇H₂₅FN₂O (412.50): calcd. C, 78.62; H, 6.11; N, 6.79. found C, 78.56, H, 6.16, N, 6.72.

### Example 19

### 8-(1-((5-Phenylpyridin-3-yl)methyl)piperidin-4-yl)quinolin-2(1H)-one (Compound 4c)

Following the same procedure adopted for the synthesis of 3a, the title compound was obtained by reductive amination of compound 4 and 6c as a light yellow solid (yield 38%). M.p. 158-160° C.—IR (neat): ν=3363, 3051, 3018, 2932, 1643, 1600, 1433, 1208 cm⁻¹.—¹H NMR (500 MHz, CDCl₃): δ=1.90 (m, 4H, piperidine H), 2.17 (m, 2H, piperidine H), 3.07 (m, 3H, piperidine H), 3.71 (s, 2H, NCH₂), 6.59 (d, J=9.5 Hz, 1H, 3-H), 7.21 (t, J=7.6 Hz, 1H, aromatic H), 7.41-7.43 (m, 2H, aromatic H), 7.47-7.51 (m, 4H, aromatic H), 7.64 (m, 2H, aromatic H), 7.7 (d, J=9.4 Hz, 1H, 4-H) 7.92 (s, 1H, aromatic H), 8.57 (d, J=1.8 Hz, 1H, aromatic H), 8.78 (d, J=1.6 Hz, 1H, aromatic H), 10.52 (br. s, 1H, NHCO).—¹³C NMR (125.7 MHz, CDCl₃): δ=32.28, 34.67, 53.84 (all Cₚᵢₚₑᵣ), 60.47 (NCH₂), 120.03, 121.06, 122.55, 126.26, 127.19, 127.81, 128.07, 129.02, 131.34, 133.55, 135.16, 135.69, 136.31, 137.71, 141.73, 147.06, 149.16 (all Cₐᵣₒₘ) 163.65 (C═O). Anal. Calcd for C₂₆H₂₅N₃O (395.50): C, 78.96; H, 6.37; N, 10.62%. Found: C, 78.90; H, 6.43; N, 10.57%.

### Example 20

### 8-(1-((5-(4-Fluorophenyl)pyridin-3-yl)methyl)piperidin-4-yl)quinolin-2(1H)-one (Compound 4d)

Following the same procedure adopted for the synthesis of 3a, the title compound was obtained by reductive amination of compound 4 and 6d as a light yellow solid (yield 34%). M.p. 161-163° C.—IR (neat): ν=3373, 3058, 3040, 2928, 1640, 1602, 1430, 1228 cm⁻¹.—¹H NMR (500 MHz, CDCl₃): δ=1.91 (m, 4H, piperidine H), 2.39 (m, 2H, piperidine H), 3.09 (m, 3H, piperidine H), 3.71 (s, 2H, NCH₂), 6.62 (d, J=11.8 Hz, 1H, 3-H), 7.18-7.22 (m, 4H, aromatic H), 7.44-7.48 (m, 2H, aromatic H), 7.59-7.62 (m, 2H, aromatic H), 7.80 (d, J=11.2 Hz, 1H, 4-H), 7.91 (br. s, 1H, NH), 8.59 (d, J=1.8 Hz, 1H, aromatic H), 8.75 (d, J=1.8 Hz, 1H, aromatic H), 10.20 (br. s, 1H, NHCO).—¹³C NMR (125.7 MHz, CDCl₃): =32.26, 34.89, 53.99 (all Cₚᵢₚₑᵣ)_{,} 60.46 (NCH₂), 115.97, 116.18, 120.07, 121.15, 123.63, 126.38, 127.85, 128.89, 131.10, 133.87, 135.03, 135.48, 141.77, 147.01, 149.24, 163.50, 164.36 (all Cₐᵣₒₘ).—C₂₆H₂₄FN₃O (413.49): calcd. C, 75.52; H, 5.85; N, 10.16. found C, 75.46, H, 5.91, N, 10.09.

### Example 21

### 8-(1-(3-Cyclopentenylbenzyl)piperidin-4-yl)quinolin-2(1H)-one (Compound 4e)

Following the same procedure adopted for the synthesis of 3a, the title compound was obtained by reductive amination of compound 4 and 6e as a white solid (yield 42%). M.p. 123-125° C.—IR (neat): ν=3164, 3110, 3022, 3011, 2936, 2886, 1639, 1599, 1471, 1116 cm⁻¹.—¹H NMR (500 MHz, CDCl₃): δ=1.83-191 (m, 6H, piperidine H, cyclopent H), 2.21-2.29 (m, 2H, cyclopent H), 2.51-2.59 (m, 2H, piperidine H), 2.73 (m, 2H, cyclopent H), 2.83 (m, 1H, piperidine H), 3.08 (m, 2H, piperidine H), 3.60 (s, 2H, NCH₂), 6.22 (s, 1 H, cyclopent H), 6.60 (d, J=9.4 Hz, 1H, 3-H), 7.11-7.28 (m, 2H, aromatic H), 7.29 (t, J=7.8 Hz, 1H, aromatic H), 7.33 (m, 1H, aromatic H), 7.42 (m, 2H, aromatic H), 7.52 (m, 1H, aromatic H), 7.75 (d, J=9.4 Hz, 1H, 4-H), 9.36 (br. s, 1H, NHCO).—¹³C NMR (125.7 MHz, CDCl₃): δ=23.37 (C_{cyclopent}), 32.27 (Cₚᵢₚₑᵣ), 33.28, 33.34 (all C_{cyclopent}), 35.47, 53.99 (all Cₚᵢₚₑᵣ), 63.44 (NCH₂), 119.94, 121.21, 122.58, 124.37, 126.26, 126.43, 127.81, 128.18, 130.84, 135.47, 136.77, 137.01, 141.64, 142.42 (all Cₐᵣₒₘ), 162.98 (C═O).—C₂₆H₂₈N₂O (384.51): calcd. C, 81.21; H, 7.34; N, 7.29. found C, 81.15; H, 7.40; N, 7.22.

### Example 22

### 8-(1-((5-Cyclopentenylpyridin-3-yl)-methyl)piperidin-4-yl)quinolin-2(1H)-one (Compound 4f)

Following the same procedure adopted for the synthesis of 3a, the title compound was obtained by reductive amination of compound 4 and 6f as an off white solid (yield 31%). M.p. 133-135° C.—IR (neat): ν=3169, 3026, 2934, 1639, 1601, 1411, 1190, 1127 cm⁻¹.—¹H NMR (500 MHz, CDCl₃): δ=1.81-191 (m, 4H, piperidine H), 1.98-2.10 (m, 2H, cyclopent H), 2.25-2.36 (m, 2H, cyclopent H), 2.52-2.59 (m, 2H, piperidine H), 2.72 (m, 2H, cyclopent H), 3.00 (m, 3H, piperidine H), 3.59 (s, 2H, NCH₂), 6.30 (s, 1H, cyclopent H), 6.57 (d, J=9.4 Hz, 1H, 3-H), 7.18 (t, J=8.8 Hz, 1H, aromatic H), 7.41 (m, 2H, aromatic H), 7.68 (s, 1H, aromatic H), 7.76 (d, J=9.4 Hz, 1H, 4-H), 8.40 (s, 1H, aromatic H), 8.59 (s, 1H, aromatic 1), 10.18 (br. s, 1H, NHCO).—¹³C NMR (125.7 MHz, CDCl₃): δ=23.33 (C_{cyclopent}), 32.33 (Cₚᵢₚₑᵣ), 33.22, 33.30 (all C_{cyclopent}), 36.32 (Cₚᵢₚₑᵣ), 53.05 (OCH₃), 54.74 (Cₚᵢₚₑᵣ), 63.62 (NCH₂), 113.98 (C_{cyclopent}), 120.14 (Ar—C), 122.26, 122.41, 124.11, 128.50, 132.05, 132.59, 133.36, 133.58, 138.80, 139.29, 140.65, 145.98, 148.45 (all Cₐᵣₒₘ), 162.28 (C-2).—C₂₅H₂₇N₃O (385.50): calcd. C, 77.89; H, 7.06; N, 10.90. found C, 77.83; H, 7.12; N, 10.83.

### Example 23

### 8-(1-(Biphenyl-4-ylmethyl)piperidin-4-yl)-3,4-dihydroquinolin-2(1H)-one (Compound 5a)

Following the same procedure adopted for the synthesis of 3a, the title compound was obtained by reductive amination of compound 5 and 6a as an off white solid (yield 41%). M.p. 116-118° C.—IR (neat): ν=3217, 3053, 2912, 2872, 1668, 1601, 1482, 1211 cm⁻¹.—¹H NMR (500 MHz, CDCl₃): δ=1.76-184 (m, 4H, piperidine H), 2.15 (m, 4 II, piperidine H), 2.53 (m, 1H, piperidine H), 2.59 (t, J=7.6, 2H, 4-H), 2.94 (t, J=6.7, 2H, 341), 3.05 (m, 2H, piperidine H), 3.60 (s, 2H, NCH₂), 6.97-7.03 (m, 2H, aromatic H), 7.15 (m, 1H, aromatic H), 7.34 (m, 1H, aromatic H), 7.41-7.45 (m, 4H, aromatic H), 7.55 (d, J=7.3, 1H, aromatic H), 7.60 (d, J=7.3, 1H, aromatic H), 7.87 (br. s, 1H, NHCO).—¹³C NMR (125.7 MHz, CDCl₃): δ=25.95 (C-4), 30.62 (C-3), 32.24, 35.82, 54.07 (all Cₚᵢₚₑᵣ), 63.00 (NCH₂), 123.09, 124.21, 124.80, 125.69, 126.92, 127.01, 127.12, 128.69, 129.62, 130.91, 134.20, 137.22, 139.95, 140.91 (all Cₐᵣₒₘ), 171.68 (C-2). C₂₇H₂₈N₂O (396.52): calcd. C, 81.78, H, 7.12, N, 7.06. found C, 81.72; H, 7.17; N, 7.00.

### Example 24

### 8-(1-((4′-Fluorobiphenyl-4-yl)methyl)piperidin-4-yl)-3,4-dihydroquinolin-2(1H)-one (Compound 5b)

Following the same procedure adopted for the synthesis of 3a, the title compound was obtained by reductive amination of compound 5 and 6b as an light yellow solid (yield 33%). M.p. 132-134° C.—IR (neat): ν=3223, 3050, 2902, 2862, 1667, 1600, 1489, 1231 cm⁻¹. —¹H NMR (500 MHz, CDCl₃): δ=1.67-173 (m, 4H, piperidine H), 2.07 (m, 2H, piperidine H), 2.50 (m, 3H, piperidine H, 4-H), 2.83 (t, J=7.6, 2H, 3-H), 2.96 (m, 2H, piperidine H), 3.51 (s, 2 II, NCH₂), 6.88-6.94 ((m, 2H, aromatic H), 7.01-7.12 ((m, 3H, aromatic H), 7.32 (d, J=7.6 Hz, 2H, aromatic H), 7.39-7.49 ((m, 4H, aromatic H), 7.94 (br. s, 1H, NHCO).—¹³C NMR (125.7 MHz, CDCl₃): δ=26.02 (C-4), 30.71 (C-3), 32.30, 35.78, 54.10 (all Cₚᵢₚₑᵣ), 63.02 (NCH₂), 115.52, 115.73, 123.16, 124.31, 124.87, 125.87, 126.84, 128.56, 129.78, 131.05, 134.31, 137.08, 137.24, 139.04, 161.32, 163.62 (all Cₐᵣₒₘ,), 170.87 (C-0). C₂₇H₂₇FN₂O (414.51): calcd. C, 78.23; H, 6.57; N, 6.76. found C, 78.26, II 6.62, N, 6.69.

### Example 25

### 8-(1-((5-Phenylpyridin-3-yl)methyl)piperidin-4-yl)-3,4-dihydroquinolin-2(1H)-one (Compound 5c)

Following the same procedure adopted for the synthesis of 3a, the title compound was obtained by reductive amination of compound 5 and 6c as a light yellow gum (yield 34%).—IR (neat): ν=3213, 3032, 2922, 1662, 1608, 1472, 1201 cm⁻¹.—¹H NMR (500 MHz, CDCl₃): δ=1.76-189 (m, 4H, piperidine H), 2.27 (m, 2H, piperidine H), 2.56-2.66 (m, 3H, piperidine H, 4-H), 2.93 (m, 2H, 3-H), 3.05 (m, 2H, piperidine H), 3.67 (s, 2H, NCH₂), 6.97 (m, 2H, aromatic H), 7.07 (m, 1H, aromatic H), 7.43 (m, 1H, aromatic H), 7.50 (t, J=7.5 Hz, 2H, aromatic H), 7.89 (s, 1H, aromatic H), 8.41 (s, 1H, aromatic H), 8.54 (s, 1H, NHCO), 8.72 (s, 1H, aromatic H).—¹³C NMR (125.7 MHz, CDCl₃): δ=25.93 (C-4), 30.63 (C-3), 31.93, 35.26, 53.85 (all Cₚᵢₚₑᵣ), 60.13 (s, 2H, NCH₂), 115.99, 116.20, 123.34, 124.43, 124.91, 125.96, 128.87, 128.95, 130.99, 133.68, 134.21, 135.43, 146.80, 148.93, 161.76, 164.22 (all _{Carom})_{,} 172.50 (C═O). C₂₆H₂₇N₃O (397.51): calcd. C, 78.56; H, 6.85; N, 10.57. found C, 78.50; H, 6.91; N, 10.50.

### Example 26

### 8-(1-((5-(4-Fluorophenyl)pyridin-3-yl)methyl)piperidin-4-yl)-3,4-dihydroquinolin-2(1H)-one (Compound 5d)

Following the same procedure adopted for the synthesis of 3a, the title compound was obtained by reductive amination of compound 5 and 6d as a light yellow solid (yield 30%). M.p. 136-138° C.—IR (neat): ν=3198, 3051, 2931, 1660, 1608, 1468, 1186 cm⁻¹.—¹H NMR (500 MHz, CDCl₃): δ=1.74-189 (m, 4H, piperidine H), 2.31 (m, 2H, piperidine H), 2.56 (m, 2H, 4-H), 2.72 (m, 1H, piperidine H), 2.89 (m, 2H, 3-H), 3.07 (m, 2H, piperidine H), 3.70 (s, 2H, NCH₂), 6.93-7.06 (m, 2H, aromatic H), 7.13 (d, J=8.2 Hz, 1H, aromatic H), 7.35-7.55 (t, J=9.0 Hz, 3H, aromatic H), 7.62 (m, 2 II, aromatic H), 7.93 (s, 1H, aromatic H), 8.54 (s, 1H, aromatic H), 9.61 (br. s, 1H, NHCO).—¹³C NMR (125.7 MHz, CDCl₃): δ=25.95 (C-4), 30.68 (C-3), 31.95, 35.04, 53.69 (all Cₚᵢₚₑᵣ), 60.07 (NCH₂), 123.30, 124.46, 124.93, 125.90, 127.20, 128.23, 129.10, 131.25, 133.00, 134.37, 135.69, 136.55, 137.51, 146.91, 148.90 (all Cₐᵣₒₘ), 172.66 (C═O). C₂₆H₂₆FN₃O (415.50): calcd. C, 75.16, H, 6.31, N, 10.11. found C, 75.10; H, 6.37; N, 10.03.

### Example 27

### 8-(1-(3-Cyclopentenylbenzyl)piperidin-4-yl)-3,4-dihydroquinolin-2(1H)-one (Compound 5e)

Following the same procedure adopted for the synthesis of 3a, the title compound was obtained by reductive amination of compound 5 and 6e as a light green solid (yield 45%). M.p. 115-117° C.—IR (neat): ν=3191, 3067, 2922, 2842, 1665, 1603, 1431, 1188 cm⁻¹.—¹H NMR (500 MHz, CDCl₃): δ=1.71-189 (m, 4H, piperidine H), 1.98-2.08 (m, 2H, cyclopent H), 2.11-2.18 (m, 2H, cyclopent H), 2.48-2.56 (m, 5H, piperidine H, 4-H), 2.74 (m, 2H, cyclopent H), 2.95 (m, 2H, 3-H), 3.03 (m, 2H, piperidine H), 3.57 (s, 2H, NCH₂), 6.22 (br. s, 1H, cyclopent H), 6.94-7.13 (m, 2H, aromatic H), 7.16 (d, J=7.7 Hz, 1H, aromatic H), 7.23 (d, J=7.6 Hz, 1H, aromatic H), 7.25-7.32 (m, 2H, aromatic H), 7.41 (s, 1 H, aromatic H), 7.98 (br. s, 1H, NHCO).—¹³C NMR (125.7 MHz, CDCl₃): δ=23.40 (C_{cyclopent}), 26.04 (C-4), 30.71 (C-3), 32.31 (Cₚᵢₚₑᵣ), 33.29, 33.37 (all C_{cyclopent}), 35.83, 54.07 (all Cₚᵢₚₑᵣ), 63.49 (NCH₂), 123.13 (C_{cyclopent})_{,} 124.28, 124.37, 124.88, 125.83, 126.27, 126.26, 127.88, 128.18, 131.11, 134.30, 136.82, 137.04, 142.42 (all Cₐᵣₒₘ), 171.82 (C═O). C₂₆H₃₀N₂O (386.53): calcd. C, 80.79; H, 7.82; N, 7.25. found C, 80.73; H, 7.88; N, 7.18.

### Example 28

### 8-(1-((5-cyclopentenylpyridin-3-yl)methyl)piperidin-4-yl)-3,4-dihydroquinolin-2(1H)-one (Compound 5f)

Following the same procedure adopted for the synthesis of 3a, the title compound was obtained by reductive amination of compound 5 and 6f as a light yellow solid (yield 39%). M.p. 123-125° C.—IR (neat): ν=3195, 3057, 2932, 2832, 1667, 1600, 1437, 1182 cm⁻¹.—¹H NMR (500 MHz, CDCl₃): δ=1.73-188 (m, 4H, piperidine H), 2.00-2.11 (m, 2H, cyclopent H), 2.12-2.16 (m, 2H, cyclopent H), 2.51-2.66 (m, 4H, piperidine H, 441), 2.69-2.76 (m, 2H, cyclopent H), 2.92-3.06 (m, 4H, piperidine H, 3-H), 3.57 (s, 2H, NCH₂), 6.31 (br. s, 1H, cyclopent H), 6.92-7.12 (m, 2H, aromatic H), 7.16 (d, J=7.7 Hz, 1H, aromatic H), 7.69 (s, 1H, aromatic H), 8.12 (br. s, 1H, NHCO), 8.41 (s, 1H, aromatic H), 8.59 (s, 1H, aromatic H).—¹³C NMR (125.7 MHz, CDCl₃): δ=23.25 (C_{cyclopent}), 26.03 (C-4), 30.71 (C-3), 32.25 (Cₚᵢₚₑᵣ), 32.99, 33.44 (all C_{cyclopent}), 35.62, 54.04 (all Cₚᵢₚₑᵣ), 60.57 (NCH₂), 123.13 (C_{cyclopent}), 124.34, 124.84, 125.88, 128.35, 130.98, 132.02, 133.07, 133.31, 134.33, 139.49, 146.02, 148.68 (all Cₐᵣₒₘ), 171.87 (C═O). C₂₅H₂₉N₃O (387.52): calcd. C, 77.48, H, 7.54, N, 10.84. found C, 77.42; H, 7.60; N, 10.77.

It is to be understood that the present invention is not limited to the embodiments described above, but encompasses any and all embodiments within the scope of the following claims.

We claim:

1. A 4-aryl-1-(biarylmethylene) piperidine compound having the formula: 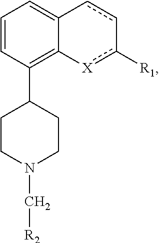
wherein R₁ is methoxy or oxo; X is N or NH; R₂ is a biaryl substituent selected from the group consisting of biphenyl, fluorophenyl benzene, 3-phenyl pyridine, 3-(4-fluorophenyl) pyridine, phenyl cyclopentene, and 3-(1-cyclopenten-1-yl) pyridine; and the dashed lines (------) represent either a single or a double bond; and pharmaceutically acceptable salts thereof.

2. The 4-aryl-1-(biarylmethylene)piperidine compound according to claim 1, wherein the compound has the formula: 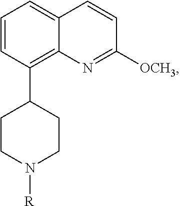
wherein R is selected from the group consisting of:

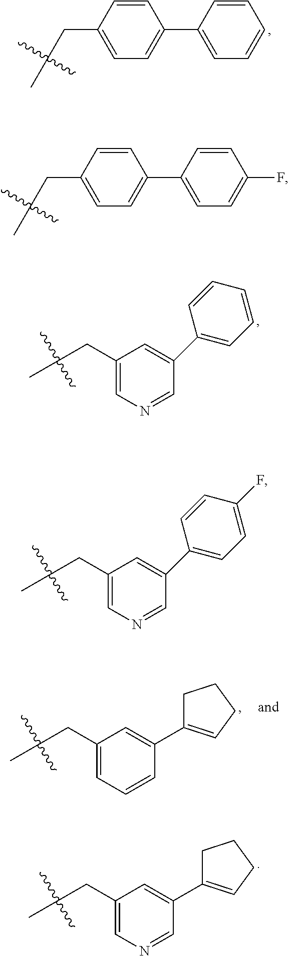 [SMILES: *Cc1ccc(-c2ccccc2)cc1 ; *Cc1ccc(-c2ccc(F)cc2)cc1 ; *Cc1cncc(-c2ccccc2)c1 ; *Cc1cncc(-c2ccc(F)cc2)c1 ; *Cc1cccc(C2=CCCC2)c1 ; *Cc1cncc(C2=CCCC2)c1]

3. The 4-aryl-1-(biarylmethylene)piperidine compound according to claim 1, wherein the compound has the formula: 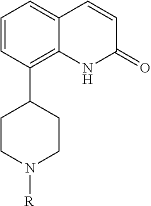
wherein R is selected from the group consisting of:

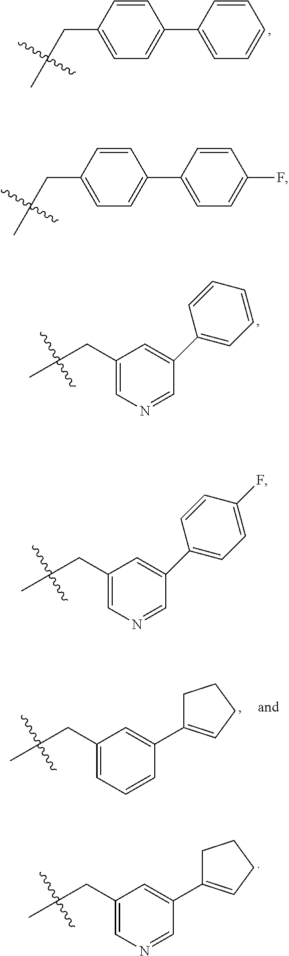 [SMILES: *Cc1ccc(-c2ccccc2)cc1 ; *Cc1ccc(-c2ccc(F)cc2)cc1 ; *Cc1cncc(-c2ccccc2)c1 ; *Cc1cncc(-c2ccc(F)cc2)c1 ; *Cc1cccc(C2=CCCC2)c1 ; *Cc1cncc(C2=CCCC2)c1]

4. The 4-aryl-1-(biarylmethylene)piperidine compound according to claim 1, wherein the compound has the formula: 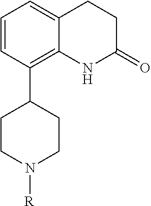
wherein R is selected from the group consisting of: 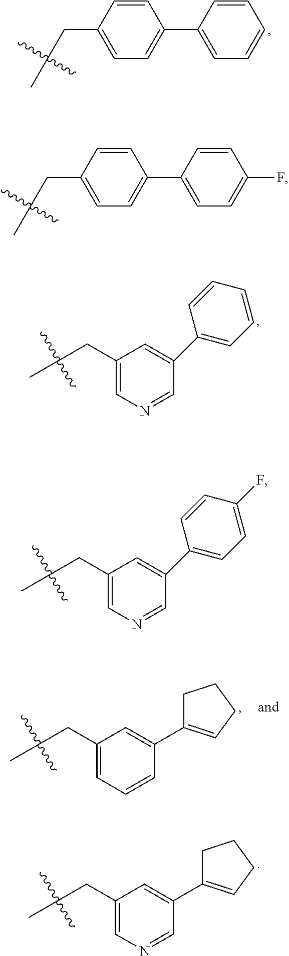 [SMILES: *Cc1ccc(-c2ccccc2)cc1 ; *Cc1ccc(-c2ccc(F)cc2)cc1 ; *Cc1cncc(-c2ccccc2)c1 ; *Cc1cncc(-c2ccc(F)cc2)c1 ; *Cc1cccc(C2=CCCC2)c1 ; *Cc1cncc(C2=CCCC2)c1]
